(19) United States
(12) Patent Application Publication
(10) Pub. No.: US 2022/0212235 A1
(43) Pub. Date: Jul. 7, 2022

(54) Paper Currency Sanitizing Assembly

(71) Applicant: Douglas Martin, Carson, CA (US)

(72) Inventor: Douglas Martin, Carson, CA (US)

(21) Appl. No.: 17/141,359

(22) Filed: Jan. 5, 2021

(51) Int. Cl. B08B 7/00; B65H 37/00; A61L 2/10

(52) U.S. Cl. CPC B08B 7/0057; B65H 37/00; B65H 2701/1912; A61L 2202/11; A61L 2202/14; A61L 2/10

(57) ABSTRACT

A paper currency sanitizing assembly includes a housing that has an entry and an exit to pass paper currency therethrough. A conveyor belt is rotatably integrated into the housing to transport the paper currency between the entry and the exit. The conveyor belt is comprised of a translucent material to pass light therethrough. A set of first light emitters is each positioned within the housing to emit light onto the conveyor belt. A set of second light emitters is each of the second light emitters is positioned within the housing to emit light onto the conveyor belt. Moreover, each of the first light emitters and the second has an operational frequency in the spectrum of ultra-violet light to kill bacteria on the paper currency.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sanitizing devices and more particularly pertains to a new sanitizing device for sanitizing paper currency. The sanitizing device includes a translucent conveyor belt and ultraviolet light emitters positioned above and below the conveyor belt. In this way the ultraviolet light emitters sterilize the paper currency.

### (2) Description of Related Art Including Information Disclosed Under 37 CFR 1.97 and 1.98

The prior art relates to sanitizing devices including a variety of sanitizing devices for sanitizing paper currency which includes conveyor rollers and ultraviolet light. In at least one instance a liquid sanitizer is applied to the paper currency. Additionally, the prior art discloses a cash drawer that is exposed to ultraviolet light when the cash drawer is closed to sanitize paper currency in the cash drawer. In no instance does the prior art disclose a translucent conveyor belt for passing ultraviolet light therethrough to sanitize paper currency.

### BRIEF SUMMARY OF THE INVENTION

An embodiment of the disclosure meets the needs presented above by generally comprising a housing that has an entry and an exit to pass paper currency therethrough. A conveyor belt is rotatably integrated into the housing to transport the paper currency between the entry and the exit. The conveyor belt is comprised of a translucent material to pass light therethrough. A set of first light emitters is each positioned within the housing to emit light onto the conveyor belt. A set of second light emitters is each of the second light emitters is positioned within the housing to emit light onto the conveyor belt. Moreover, each of the first light emitters and the second has an operational frequency in the spectrum of ultra-violet light to kill bacteria on the paper currency.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective in-use view of a paper currency sanitizing assembly according to an embodiment of the disclosure.

FIG. 2 is a front view of an embodiment of the disclosure.

FIG. 3 is a left side phantom view of an embodiment of the disclosure.

FIG. 4 is a right side view of an embodiment of the disclosure.

FIG. 5 is a top phantom view of an embodiment of the disclosure.

### DETAILED DESCRIPTION OF THE INVENTION

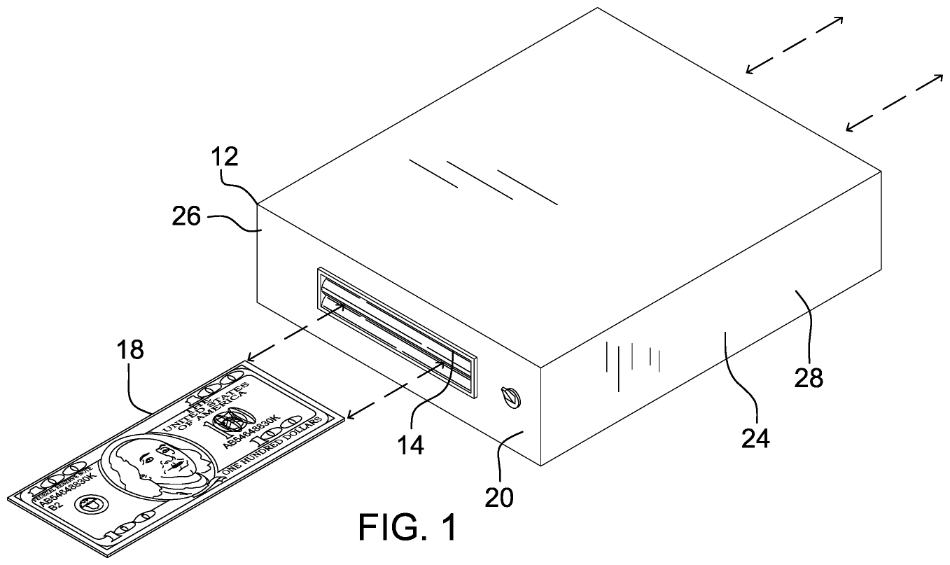

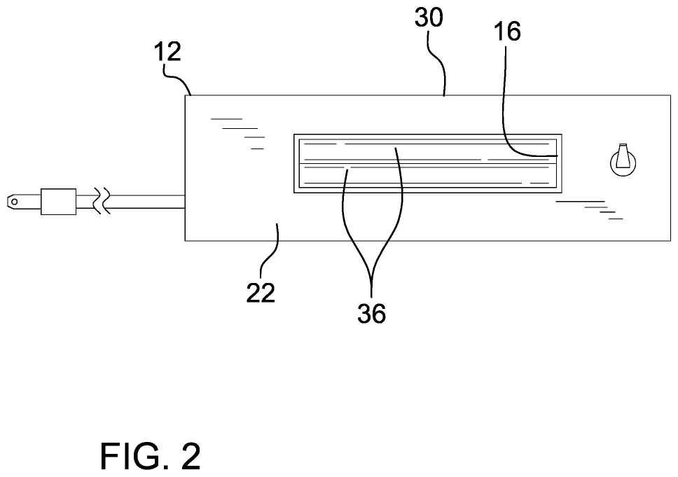

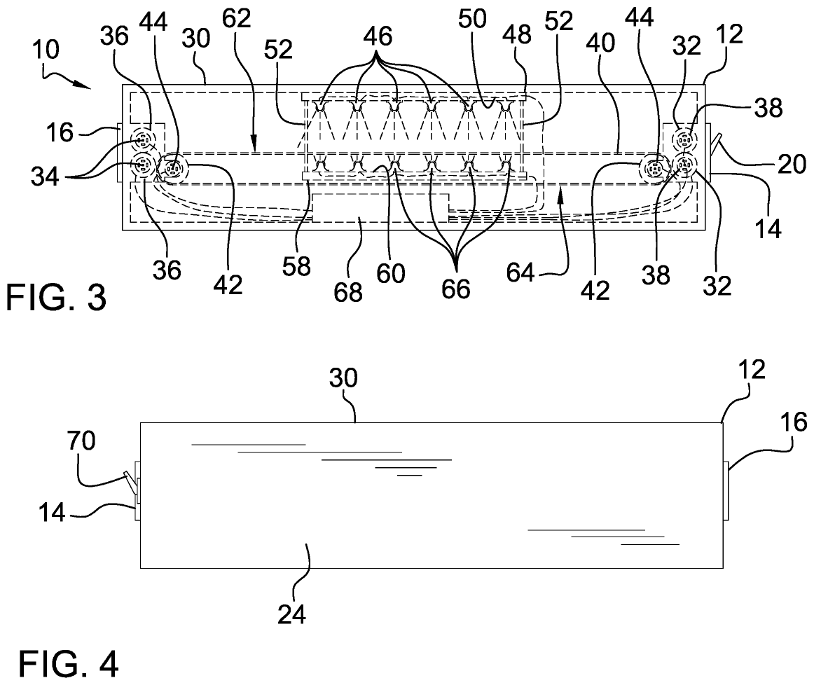

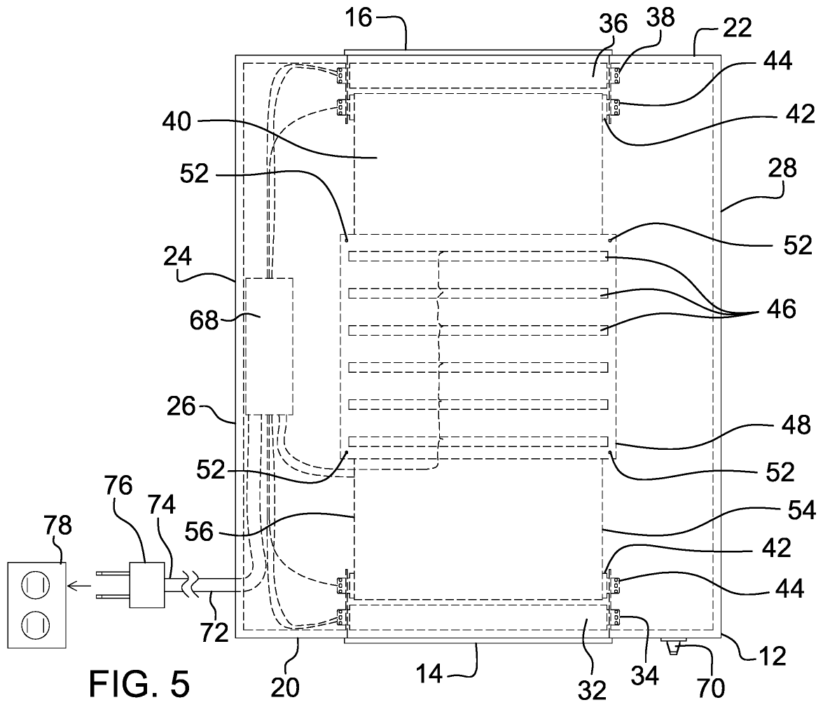

With reference now to the drawings, and in particular to FIGS. 1 through 5 thereof, a new sanitizing device embodying the principles and concepts of an embodiment of the disclosure and generally designated by the reference numeral 10 will be described.

As best illustrated in FIGS. 1 through 5, the paper currency sanitizing assembly 10 generally comprises a housing 12 that has an entry 14 and an exit 16 to pass paper currency 18 therethrough. The housing 12 has a front wall 20, a back wall 22 and an outer wall 24 extending therebetween, and the outer wall 24 has a first lateral side 26, a second lateral side 28 and a top side 30. The entry 14 extends through the front wall 20 and the exit 16 extends through the back wall 22. A pair of first rollers 32 is each rotatably integrated into the housing 12 and each of the first rollers 32 is aligned with the entry 14. The first rollers 32 are stacked on each other to urge the paper currency 18 between the first rollers 32 when the first rollers 32 are rotated. Each of the first rollers 32 extends between the first lateral side 26 and the second lateral side 28 of the outer wall 24 of the housing 12.

A pair of first motors 34 is provided and each of the first motors 34 is rotatably coupled to a respective one of the first rollers 32. Each of the first motors 34 rotates in opposing directions from each other when the first motors 34 are turned on. Additionally, each of the first rollers 32 rotate in opposite directions from each other when the first motors 34 are turned to urge the paper currency 18 inwardly through the entry 14 in the housing 12. Each of the first motors 34 may comprise an electric motor or the like.

A pair of second rollers 36 is rotatably integrated into the housing 12 and each of the second rollers 36 is aligned with the exit 16. The second rollers 36 are stacked on each other to urge the paper currency 18 between the second rollers 36 when the second rollers 36 are rotated. Moreover, each of the second rollers 36 extends between the second lateral side 28 and the second lateral side 28 of the outer wall 24 of the housing 12. A pair of second motors 38 is each rotatably coupled to a respective one of the second rollers 36. Each of the second motors 38 rotates in opposing directions from each other when the second motors 38 are turned on. Additionally, each of the second rollers 36 rotates in opposite directions from each other when the second motors 38 are turned on to urge the paper currency 18 outwardly through the entry 14 in the housing 12. Each of the second motors 38 may comprise an electric motor or the like.

A conveyor belt 40 is rotatably integrated into the housing 12 and the conveyor belt 40 extends between the first rollers 32 and the second rollers 36. In this way the conveyor belt 40 can transport the paper currency 18 between the first rollers 32 and the second rollers 36. The conveyor belt 40 is comprised of a translucent material to pass light therethrough. A pair of conveyor rollers 42 is each rotatably integrated into the housing 12. Each of the conveyor rollers 42 extends between the first lateral side 26 and the second lateral side 28 of the outer wall 24 of the housing 12. Additionally, each of the conveyor rollers 42 is positioned adjacent to a respective one of the pair of first rollers 32 and the pair of second rollers 36, and the conveyor belt 40 extends around each of the conveyor rollers 42.

A pair of conveyor motors 44 is each rotatably coupled to a respective one of the conveyor rollers 42. Each of the conveyor motors 44 rotates in a first direction when the conveyor motors 44 is turned on and each of the conveyor rollers 42 is rotated in the first direction when the conveyor motors 44 are turned on. Each of the conveyor motors 44 may comprise an electric motor or the like.

A set of first light emitters 46 is each positioned within the housing 12. Each of the first light emitters 46 is directed downwardly toward the conveyor belt 40 emit light onto the conveyor belt 40. Additionally, each of the first light emitters 46 has an operational frequency in the spectrum of ultra-violet light to kill bacteria on the paper currency 18. In this way the paper currency 18 is sanitized as the conveyor belt 40 transports the paper currency 18 through the housing 12. Each of the first light emitters 46 is positioned above the conveyor belt 40 and the first light emitters 46 are spaced apart from each other and are distributed along a substantial length of the conveyor belt 40. Each of the first light emitters 46 may comprise an LED or other type of electronic light emitter.

A first panel 48 is coupled to the top side 30 of the outer wall 24 of the housing 12 and the first panel 48 has a first surface 50 that is directed toward the conveyor belt 40. Each of the first light emitters 46 is coupled to the first surface 50. A plurality of rods 52 is each coupled to and extends downwardly from the first surface 50 of the first panel 48. Each of the rods 52 is aligned with a respective one of four corners of the first panel 48 such that each of the rods 52 extends downwardly adjacent to a respective left side 54 and right side 56 of the conveyor belt 40. A second panel 58 is provided that has a primary surface 60 and each of the rods 52 is coupled to the primary surface 60. Thus, the second panel 58 is positioned between a top portion 62 and a bottom portion 64 of the conveyor belt 40 having the primary surface 60 being directed toward the top portion 62. Additionally, the paper currency 18 rests on the top portion 62 of the conveyor belt 40 while the conveyor belt 40 is transporting the paper currency 18 through the housing 12.

A set of second light emitters 66 is provided and each of the second light emitters 66 is positioned within the housing 12. Each of the second light emitters 66 is directed upwardly toward the conveyor belt 40 to emit light onto the conveyor belt 40. Moreover, each of the second light emitters 66 has an operational frequency in the spectrum of ultra-violet light to kill bacteria on the paper currency 18. In this way the paper currency 18 is sanitized while the paper currency 18 is transported through the housing 12. Each of the second light emitters 66 is positioned below the conveyor belt 40 and the second light emitters 66 are spaced apart from each other and are distributed along a substantial length of the conveyor belt 40. Moreover, each of the second light emitters 66 is coupled to the primary surface 60 of the second panel 58. Each of the second light emitters 66 may comprise an LED or other type of electronic light emitter.

A control circuit 68 is positioned in the housing 12 and the control circuit 68 is electrically coupled to each of the first motors 34. The control circuit 68 is electrically coupled to each of the second motors 38 and the control circuit 68 is electrically coupled to each of the conveyor motors 44. Additionally, the control circuit 68 is electrically coupled to each of the first light emitters 46 and the control circuit 68 is electrically coupled to each of the second light emitters 66. A power switch 70 is movably coupled to the housing 12 and the power switch 70 is electrically coupled to the control circuit 68. Each of the first motors 34, each of the second motors 38, each of the conveyor motors 44, each of the first light emitters 46 and each of the second light emitters 66 is turned on when the power switch 70 is turned on and conversely turned off when the power switch 70 is turned off.

A power cord 72 is coupled to and extends away from the housing 12 and the power cord 72 is electrically coupled to the control circuit 68. The power cord 72 has a distal end 74 with respect to the housing 12 and a male electrical plug 76 is electrically coupled to the distal end 74. The male electrical plug 76 can be plugged into a power source 72 comprising a female electrical outlet.

In use, the power switch 70 is turned on the paper currency 18 is fed into the first rollers 32. Thus, the paper currency 18 is positioned on the conveyor belt 40 to be transported through the housing 12 to be sterilized by the first light emitters 46 and the second light emitters 66. The housing 12 may be positioned near a cash register in a retail environment or any other location when paper currency 18 is commonly exchanged. The power switch 70 is turned off when all of the paper currency 18 has been fed through the housing 12 and sterilized.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A paper currency sanitizing assembly for killing bacteria on paper currency, said assembly comprising:
a housing having an entry and an exit wherein said entry and exit are each configured to pass paper currency therethrough;
a pair of first rollers, each of said first rollers being rotatably integrated into said housing, each of said first rollers being aligned with said entry, said first rollers being stacked on each other wherein said first rollers is configured to urge the paper currency between said first rollers when said first rollers are rotated;
a pair of second rollers, each of said second rollers being rotatably integrated into said housing, each of said second rollers being aligned with said exit, said second rollers being stacked on each other wherein said second rollers is configured to urge the paper currency between said second rollers when said second rollers are rotated;
a conveyor belt being rotatably integrated into said housing, said conveyor belt extending between said first rollers and said second rollers wherein said conveyor belt is configured to transport the paper currency between said first rollers and said second rollers, said conveyor belt being comprised of a translucent material wherein said conveyor belt is configured to pass light therethrough;
a set of first light emitters, each of said first light emitters being positioned within said housing, each of said light emitters being directed downwardly toward said conveyor belt wherein each of said light emitters is configured to emit light onto said conveyor belt, each of said light emitters having an operational frequency in the spectrum of ultra-violet light wherein each of said first light emitters is configured to kill bacteria on the paper currency; and
a set of second light emitters, each of said second light emitters being positioned within said housing, each of said second light emitters being directed downwardly toward said conveyor belt wherein each of said second light emitters is configured to emit light onto said conveyor belt, each of said second light emitters having an operational frequency in the spectrum of ultra-violet light wherein each of said second light emitters is configured to kill bacteria on the paper currency.

2. The assembly according to claim 1, wherein said housing has a front wall, a back wall and an outer wall extending therebetween, said outer wall having a first lateral side, a second lateral side and a top side, said entry extending through said front wall, said exit extending through said back wall.

3. The assembly according to claim 1, further comprising a pair of first motors, each of said first motors being rotatably coupled to a respective one of said first rollers, each of said first motors rotating in a opposing directions from each other when said first motors is turned on, each of said first rollers rotating in opposite directions from each other when said first motors are turned on wherein each of said first rollers is configured to urge the paper currency inwardly through said entry in said housing.

4. The assembly according to claim 1, further comprising:
a pair of first motors; and
a pair of second motors, each of said second motors being rotatably coupled to a respective one of said second rollers, each of said second motors rotating in a opposing directions from each other when said second motors is turned on, each of said second rollers rotating in opposite directions from each other when said second motors are turned on wherein each of said second rollers is configured to urge the paper currency outwardly through said entry in said housing.

5. The assembly according to claim 2, further comprising a pair of conveyor rollers, each of said conveyor rollers being rotatably integrated into said housing, each of said conveyor rollers extending between said first lateral side and said second lateral side of said outer wall of said housing, each of said conveyor rollers being positioned adjacent to a respective one of said pair of first rollers and said pair of second rollers, said conveyor belt extending around each of said conveyor rollers.

6. The assembly according to claim 5, further comprising a pair of conveyor motors, each of said conveyor motors being rotatably coupled to a respective one of said conveyor rollers, each of said conveyor motors rotating in a first direction when said conveyor motors is turned on, each of said conveyor rollers being rotated in said first direction when said conveyor motors is turned on.

7. The assembly according to claim 2, further comprising a first panel being coupled to said top side of said outer wall of said housing, said first panel having a first surface being directed toward said conveyor belt, each of said first light emitters being coupled to said first surface.

8. The assembly according to claim 7, further comprising a plurality of rods, each of said rods being coupled to and extending downwardly from said first surface of said first panel, each of said rods being aligned with a respective one of four corners of said first panel such that each of said rods extends downwardly adjacent to a respective left side and right side of said conveyor belt.

9. The assembly according to claim 8, further comprising a second panel having a primary surface, each of said rods being coupled to said primary surface such that said second panel is positioned between a top portion and a bottom portion of said conveyor belt having said primary surface being directed toward said top portion.

10. The assembly according to claim 9, wherein said second light emitters is spaced apart from each other and being distributed along a substantial length of said conveyor belt, each of said second light emitters being coupled to said primary surface of said second panel.

11. The assembly according to claim 1, further comprising:
a pair of first motors;
a pair of second motors;
a pair of conveyor motors; and
a control circuit being positioned in said housing, said control circuit being electrically coupled to each of said first motors, said control circuit being electrically coupled to each of said second motors, said control circuit being electrically coupled to each of said conveyor motors, said control circuit being electrically coupled to each of said first light emitters, said control circuit being electrically coupled to each of said second light emitters.

12. The assembly according to claim 11, further comprising a power switch being movably coupled to said housing wherein said power switch is configured to be manipulated by a user, said power switch being electrically coupled to said control circuit, each of said first motors, each of said second motors, each of said conveyor motors, each of said first light emitters and each of said second light emitters being turned on when said power switch is turned on and conversely turned off when said power switch is turned off.

13. The assembly according to claim 11, further comprising a power cord being coupled to and extending away from said housing, said power cord being electrically coupled to said control circuit, said power cord having a distal end with respect to said housing, said distal end having a male electrical plug being electrically coupled thereto wherein said male electrical plug is configured to be plugged into a power source comprising a female electrical outlet.

14. A paper currency sanitizing assembly for killing bacteria on paper currency, said assembly comprising:
a housing having an entry and an exit wherein said entry and exit are each configured to pass paper currency therethrough, said housing having a front wall, a back wall and an outer wall extending therebetween, said outer wall having a first lateral side, a second lateral side and a top side, said entry extending through said front wall, said exit extending through said back wall;
a pair of first rollers, each of said first rollers being rotatably integrated into said housing, each of said first rollers being aligned with said entry, said first rollers being stacked on each other wherein said first rollers is configured to urge the paper currency between said first rollers when said first rollers are rotated, each of said first rollers extending between said first lateral side and said second lateral side of said outer wall of said housing;
a pair of first motors, each of said first motors being rotatably coupled to a respective one of said first rollers, each of said first motors rotating in a opposing directions from each other when said first motors is turned on, each of said first rollers rotating in opposite directions from each other when said first motors are turned on wherein each of said first rollers is configured to urge the paper currency inwardly through said entry in said housing;
a pair of second rollers, each of said second rollers being rotatably integrated into said housing, each of said second rollers being aligned with said exit, said second rollers being stacked on each other wherein said second rollers is configured to urge the paper currency between said second rollers when said second rollers are rotated, each of said second rollers extending between said second lateral side and said second lateral side of said outer wall of said housing;
a pair of second motors, each of said second motors being rotatably coupled to a respective one of said second rollers, each of said second motors rotating in a opposing directions from each other when said second motors is turned on, each of said second rollers rotating in opposite directions from each other when said second motors are turned on wherein each of said second rollers is configured to urge the paper currency outwardly through said entry in said housing;
a conveyor belt being rotatably integrated into said housing, said conveyor belt extending between said first rollers and said second rollers wherein said conveyor belt is configured to transport the paper currency between said first rollers and said second rollers, said conveyor belt being comprised of a translucent material wherein said conveyor belt is configured to pass light therethrough;
a pair of conveyor rollers, each of said conveyor rollers being rotatably integrated into said housing, each of said conveyor rollers extending between said first lateral side and said second lateral side of said outer wall of said housing, each of said conveyor rollers being positioned adjacent to a respective one of said pair of first rollers and said pair of second rollers, said conveyor belt extending around each of said conveyor rollers;
a pair of conveyor motors, each of said conveyor motors being rotatably coupled to a respective one of said conveyor rollers, each of said conveyor motors rotating in a first direction when said conveyor motors is turned on, each of said conveyor rollers being rotated in said first direction when said conveyor motors is turned on;
a set of first light emitters, each of said first light emitters being positioned within said housing, each of said first light emitters being directed downwardly toward said conveyor belt wherein each of said first light emitters is configured to emit light onto said conveyor belt, each of said first light emitters having an operational frequency in the spectrum of ultra-violet light wherein each of said first light emitters is configured to kill bacteria on the paper currency, each of said first light emitters being positioned above said conveyor belt, said first light emitters being spaced apart from each other and being distributed along a substantial length of said conveyor belt;
a first panel being coupled to said top side of said outer wall of said housing, said first panel having a first surface being directed toward said conveyor belt, each of said first light emitters being coupled to said first surface;
a plurality of rods, each of said rods being coupled to and extending downwardly from said first surface of said first panel, each of said rods being aligned with a respective one of four corners of said first panel such that each of said rods extends downwardly adjacent to a respective left side and right side of said conveyor belt;
a second panel having a primary surface, each of said rods being coupled to said primary surface such that said second panel is positioned between a top portion and a bottom portion of said conveyor belt having said primary surface being directed toward said top portion;
a set of second light emitters, each of said second light emitters being positioned within said housing, each of said second light emitters being directed upwardly toward said conveyor belt wherein each of said second light emitters is configured to emit light onto said conveyor belt, each of said second light emitters having an operational frequency in the spectrum of ultra-violet light wherein each of said second light emitters is configured to kill bacteria on the paper currency, each of said second light emitters being positioned below said conveyor belt, said second light emitters being spaced apart from each other and being distributed along a substantial length of said conveyor belt, each of said second light emitters being coupled to said primary surface of said second panel;
a control circuit being positioned in said housing, said control circuit being electrically coupled to each of said first motors, said control circuit being electrically coupled to each of said second motors, said control circuit being electrically coupled to each of said conveyor motors, said control circuit being electrically coupled to each of said first light emitters, said control circuit being electrically coupled to each of said second light emitters;
a power switch being movably coupled to said housing wherein said power switch is configured to be manipulated by a user, said power switch being electrically coupled to said control circuit, each of said first motors, each of said second motors, each of said conveyor motors, each of said first light emitters and each of said second light emitters being turned on when said power switch is turned on and conversely turned off when said power switch is turned off; and
a power cord being coupled to and extending away from said housing, said power cord being electrically coupled to said control circuit, said power cord having a distal end with respect to said housing, said distal end having a male electrical plug being electrically coupled thereto wherein said male electrical plug is configured to be plugged into a power source comprising a female electrical outlet.
